(19) United States
(12) Patent Application Publication
(10) Pub. No.: US 2023/0053833 A1
(43) Pub. Date: Feb. 23, 2023

(54) PREPARATION OF HALOGENATED ALKOXYETHANE

(71) Applicant: COMMONWEALTH SCIENTIFIC AND INDUSTRIAL RESEARCH ORGANISATION, Acton (AU)

(72) Inventors: John TSANAKTSIDIS, Acton (AU); Cecily ELDRIDGE, Acton (AU); Scott COURTNEY, Scoresby (AU)

(21) Appl. No.: 17/786,360

(22) PCT Filed: Dec. 19, 2019
(86) PCT No.: PCT/AU2019/051412

§ 371 Date: Jun. 16, 2022

(51) Int. Cl. C07C 29/62

(52) U.S. Cl. CPC C07C 29/62

(57) ABSTRACT

A process for continuous preparation of halogenated alkoxyethane of general formula XClHC—CF₂OR, where X is —Cl or —F and OR is C₁₋₄ alkoxy, the process comprising a step of introducing in a flow reactor reaction components comprising (i) a compound of general formula XClC═CF₂, (ii) abase, and (iii) a C₁₋₄ alkanol, wherein a) the flow reactor comprises one or more tubular flow line(s) having an internal cross-sectional area of less than 115 mm² through which the reaction components flow as a reaction mixture, and b) the halogenated alkoxyethane is formed at least upon the reaction components mixing, with the so formed halogenated alkoxyethane flowing out of the flow reactor in a reactor effluent.

### FIELD OF THE INVENTION

The present invention relates in general to continuous preparation of halogenated alkoxyethane, and in particular to a process for continuous preparation of halogenated alkoxyethane of general formula XClHC—CF₂OR, where X is —Cl or —F and OR is C₁₋₄ alkoxy.

### BACKGROUND

Halogenated alkoxyethane compounds constitute a significant fraction of present day active pharmaceutical ingredients, not to mention agrochemicals, dyes, flame-retardants, and imaging agents.

Synthesis of halogenated alkoxyethane compounds for use as active pharmaceutical ingredients requires reproducible pharmaceutical grade compounds. Conventionally, halogenated alkoxyethane compounds are produced through batch procedures. However, the product quality per batch can be variable and the procedures can require the use of high-pressure equipment. Current batch procedures are generally plagued by poor and inhomogeneous reagent mixing, necessitating long reaction times for relatively low conversion yields. As a result, conventional synthesis of halogenated alkoxyethane compounds almost inevitably requires costly post-processing purification procedures to ensure that a pharmaceutical grade compound is produced at a commercially relevant scale.

In addition, heat control in conventional batch procedures is particularly challenging because reactions to form halogenated alkoxyethane compounds are highly exothermic. Potential formation of toxic and highly reactive intermediates and by-products (e.g. halogen halides) also pose significant safety and waste management challenges.

In contrast to conventional batch procedures, continuous production using semi-batch or semi-continuous arrangements can afford higher yield relative to conventional batch procedures. However, uncontrolled precipitation of reaction by-products imposes frequent de-clogging and cleaning of the reactor lines, which disrupts the continuity of those processes. In addition, those alternative arrangements do not fully address the challenges of conventional batch processes in terms of thermal control, safety, waste management, high reaction times, and low conversion yield.

Accordingly, there remains an opportunity to ameliorate problems and limitations associated with conventional procedures for the synthesis of halogenated alkoxyethane compounds.

### SUMMARY OF THE INVENTION

The present invention relates to a process for continuous preparation of halogenated alkoxyethane of general formula XClHC—CF₂OR, where X is —Cl or —F and OR is C₁₋₄ alkoxy, the process comprising a step of introducing in a flow reactor reaction components comprising (i) a compound of general formula XClC═CF₂, (ii) a base, and (iii) a C₁₋₄ alkanol, wherein
a) the flow reactor comprises one or more tubular flow line(s) having an internal cross-sectional area of less than 115 mm² through which the reaction components flow as a reaction mixture, and
b) the halogenated alkoxyethane is formed at least upon the reaction components mixing, with the so formed halogenated alkoxyethane flowing out of the flow reactor in a reactor effluent.

By the present invention, the reaction components can be continuously introduced into the flow reactor and converted therein into a reactor effluent containing the target halogenated alkoxyethane. The effluent continuously flows out of the reactor and is available for further processing and/or purification, if needed. The continuous nature of the process advantageously enables halogenated alkoxyethane to be produced in commercial quantities.

By comprising one or more tubular flow line(s) having an internal cross-sectional area of less than 115 mm², the flow reactor of the invention ensures high heat exchange efficiency due to high specific surface of the tubular flow lines. In addition, the specific arrangement of one or more tubular flow line(s) through which the reaction components flow as a reaction mixture affords fast and thorough mixing of the reaction components, leading to significant improvement over conventional procedures in terms of reaction time and conversion yield.

In addition, the tubular flow line(s) provide a much more controlled environment for reaction relative to conventional systems, making the flow reactor inherently safer to operate and affording the production of a purer product relative to conventional apparatuses. In that context, extreme conditions of temperature and pressure are readily implemented in the reactor of the invention to boost chemical reactivity, yet keeping full control on process parameters.

Thus, high reaction selectivity and enhanced safety can be achieved even for very fast and highly exothermic reactions involved in the formation of the target halogenated alkoxyethane. The excellent heat and mass transfer characteristics afforded by the small-section tubular flow lines, together with the fact that the reaction is resolved along the length of the reaction channel, enables a precise control of the residence time of intermediates or products by a thermal or chemical quench of the solution.

Further, the controlled environment for reaction afforded by the small-section tubular flow lines ensures that formation of hazardous chemicals can be easily controlled. Toxic substances can be readily quenched in line, thus avoiding any undesired exposures and significantly enhancing process safety.

In some embodiments, the one or more tubular flow line(s) has/have an internal cross-sectional area of less than 30 mm². For example, the one or more tubular flow line(s) may have an internal cross-sectional area of about 28 mm². Those embodiments can provide an advantageous compromise between good thermal control and safety, low reaction times, high conversion yields, and high scale-up potential for the high throughput production of pharmaceutical grade halogenated alkoxyethanes.

In some embodiments, the one or more tubular flow line(s) has/have an internal cross-sectional area of less than 5 mm², which further improves the degree of mixing of the reactor components, thus enhancing the aforementioned advantages in terms of thermal control and safety, efficient waste management, low reaction times, and high conversion yields.

According to some particularly advantageous arrangements, the one or more tubular flow line(s) have a circular internal cross-section. In some embodiments, the tubular flow line(s) having a circular internal cross-section have a diameter between 0.1 and 6 mm, for example from 0.1 and 2 mm. These arrangements are particularly effective to obtain efficient mixing of the reaction components, further reducing reaction time and increasing conversion yield.

Despite the small scale of each tubular flow line, the reactor can readily be operated with multiple tubular flow lines making the scale up to large production quantities relatively straight forward. As a result, scale-up can be performed with minimal to no re-optimisation of the reaction conditions. In this context, it can be more effective and efficient to merely “number-up” the tubular flow lines to produce a given quantity of halogenated alkoxyethane compared with developing a single macro-flow line to produce the same amount of halogenated alkoxyethane. While a process in accordance with the present invention can be performed to produce small quantities of halogenated alkoxyethane (e.g. fraction of grams per day) by using one flow line, the flow lines can be readily “numbered-up” to produce more commercially relevant amounts of halogenated alkoxyethane (e.g. from several grams to several kilos per day), yet maintaining identical standards of safety, product purity, reaction time, reaction yield, and safety.

The process of the invention is also particularly advantageous for the production of commercially relevant halogenated alkoxyethane compounds.

For example, in some embodiments the compound of general formula XClC═CF₂ is Cl₂C═CF₂. In those instances, the process of the invention allows for the efficient and scalable production of halogenated alkoxyethane compounds such as methoxyflurane (Cl₂HC—CF₂OCH₃), which can be obtained when the C₁₋₄ alkanol is methanol. Given its high reaction yield, the process can afford facile and large-scale synthesis of pharmaceutical grade methoxyflurane.

In some embodiments, the compound of general formula XClC═CF₂ is FClC═CF₂. In those instances, the process of the invention affords efficient and scalable production of ClFHC—CF₂OCH₃, which can be obtained when the C₁₋₄ alkanol is methanol. The possibility to produce highly pure and high amounts of ClFHC—CF₂OCH₃ can be particularly advantageous, since that compound is a known precursor in the synthesis of 2-chloro-1,1,2,-trifluoroethyl-difluoromethyl ether (enflurane).

Further aspects and embodiments of the invention are discussed in more detail below.

### BRIEF DESCRIPTION OF THE DRAWINGS

The invention will also be described herein with reference to the following non-limiting drawings in which:

FIG. 1 shows an example of a suitable setup for the continuous production of halogenated alkoxyethane compounds in accordance with an embodiment of the present invention,

FIG. 2 shows a cut-away side view of a tubular flow line arrangement according to an embodiment of the present invention,

FIG. 3 shows a schematic of a suitable setup for the continuous production of halogenated alkoxyethane compounds in accordance with an embodiment of the present invention,

FIG. 4 shows a gas chromatography spectrum of the reactor effluent/water mixture obtained as sample 1 according to the procedure described in Example 5,

FIG. 5 shows a gas chromatography spectrum of the reactor effluent/water mixture obtained as sample 2 according to the procedure described in Example 5, and

FIG. 6 shows a gas chromatography spectrum of the reactor effluent/water mixture obtained as sample 3 according to the procedure described in Example 5.

### DETAILED DESCRIPTION OF THE INVENTION

The process of the invention is one for continuous preparation of halogenated alkoxyethane of general formula XClHC—CF₂OR, where X is —Cl or —F and OR is C₁₋₄ alkoxy.

As used herein, the expression “C₁₋₄ alkoxy” denotes a straight chain or branched alkoxy group having from 1 to 4 carbons. Examples of straight chain and branched alkoxy include methoxy, ethoxy, n-propoxy, isopropoxy, n-butoxy, sec-butoxy, and t-butoxy.

In some embodiments, X is —Cl and OR is a methoxy group, in which case the halogenated alkoxyethane has a formula Cl₂HC—CF₂OCH₃ (methoxyflurane).

In some embodiments, X is —F and OR is a methoxy group, in which case the halogenated alkoxyethane has a formula FClHC—CF₂OCH₃. Such compound is a known precursor for the synthesis of 2-chloro-1,1,2,-trifluoroethyl-difluoromethyl ether (enflurane).

The process of the invention is one for the continuous preparation of halogenated alkoxyethane, and is based on the use of a continuous flow reactor. By the preparation being “continuous” is meant that the halogenated alkoxyethane forms continuously as the reactor components are mixed and flow through the tubular flow lines. As such, the so-formed halogenated alkoxyethane can be collected from the effluent that exits the flow reactor continuously.

By a “flow reactor” is meant that the reactor is designed to enable (1) continuous introduction of the reaction components into the one or more tubular flow line(s) through which they flow as a reaction mixture, and (2) continuous flow out of the reactor of an effluent containing the halogenated alkoxyethane.

The flow reactor used in the process of the invention comprises one or more tubular flow line(s). By the expression “tubular flow line” is meant an elongated hollow tube that allows internal fluid flow along its main length.

In some embodiments, the flow reactor comprises one tubular flow line. In those embodiments, a singular tubular flow line connects an inlet and an outlet of the flow reactor.

In some embodiments, the flow reactor comprises two or more tubular flow lines. For example, the flow reactor may comprise two or more tubular flow lines in a parallel-flow arrangement. By “parallel-flow” arrangement is meant that fluid enters each tubular flow line at the same end of the reactor, flows within each line along the same direction, and leaves each tubular flow line at the same end of the reactor. Advantageously, these embodiments enable the straightforward scale-up of reactor output by merely increasing the number of tubular flow lines, with no need to re-optimise reaction conditions. In those embodiments, the flow reactor may have a single inlet and a single outlet for the introduction of the reactor components (or the reaction mixture) and extraction of the reactor effluent, respectively. The reactor may therefore be provided with an internal arrangement that enables the fluid flowing from the single inlet to be distributed/subdivided across each single flow line, then collected at the other end to exit the reactor as a single effluent line.

The tubular flow line (or each one of the two or more tubular flow lines) has an internal cross-sectional area of less than 115 mm². For example, the tubular flow line(s) may have an internal cross-sectional area of less than about 100 mm², less than about 50 mm², less than 25 mm², less than 10 mm², or less than 5 mm². For avoidance of doubt, by “internal” cross-sectional area is meant herein the cross-sectional area through which a fluid flows within the tubular flow line.

In some embodiments, the tubular flow line (or each one of the two or more tubular flow lines) has an internal cross-sectional area of less than about 30 mm². For example, the tubular flow line (or each one of the two or more tubular flow lines) may have an internal cross-sectional area of from about 0.2 mm² to about 30 mm². In some embodiments, the one or more tubular flow line(s) have an internal cross-sectional area of about 28 mm². Those dimensions provide a particularly advantageous combination of effective mixing of the reaction mixture and specific surface area for effective thermal control. For example, tubular flow line(s) of any of those sizes are sufficiently large to accommodate a static mixer, yet provide an adequately large specific surface area for effective thermal control. The resulting reactor represents therefore an advantageous platform for scale-up.

As used herein, the term “about”, when referring to a value or to an amount of mass, weight, time, volume, concentration, percentage, and the like can encompass variations of, and in some embodiments, ±20%, in some embodiments ±10%, in some embodiments ±5%, in some embodiments ±1%, in some embodiments ±0.5%, and in some embodiments ±0.1%, from the specified amount.

Advantageously, the continuous synthesis of halogenated alkoxyethane in one or more tubular flow line(s) of the kind described herein is more efficient than a corresponding synthesis performed in batch system according to conventional procedures. In that regard, fluid behaviour in a fluidic system of the kind described herein differs significantly from fluid behaviour in macroscopic environments. While fluid dynamics in macroscopic environments is mostly dominated by pressure and gravity, in the flow reactor of the invention surface tension, energy dissipation and fluidic resistance start to determine the fluid dynamics. In addition, mixing efficiency afforded by the one or more tubular flow line(s) of the kind described herein is superior to that of conventional processes.

It is also particularly advantageous to use a tubular flow line(s) having an internal cross-sectional area of less than 5 mm². When the tubular flow line(s) has an internal cross-sectional area of less than 5 mm², homogeneity of the reaction mixture flowing through the line(s) is advantageously high due to increased mixing efficiency of the reaction components achievable within the flow line. In addition, by having a small cross-sectional area, the tubular flow line(s) are characterised by a significantly high surface-to-volume ratio. As a result, heat transfer (and therefore thermal control) is facilitated.

Provided the internal cross-sectional area of the tubular flow line(s) is less than 115 mm², the internal cross-sectional area may have any geometry. Examples of suitable geometries of the internal cross-sectional area include a circular geometry, a square geometry, a rectangular geometry, a triangular geometry, or other geometries known in the art. This may be achieved by using round tubing and/or square tubing of the kind that would be known to the skilled person.

In some embodiments, the tubular flow line(s) has/have a circular internal cross-section geometry. By having a “circular” geometry, the internal cross-section of the line(s) has a round shape characterised by an average internal diameter. For example, the internal cross-section may have the shape of a circle.

The average internal diameter of a tubular flow line that forms such flow reactors may range between 0.1 and 12 mm. The reaction tube diameter (internal) may typically be greater than or equal to 0.2 mm but less than 12 mm (and including any integer there between, and/or fraction thereof, for example, 0.2 mm, 0.3 mm, 0.4 mm, 0.5 mm, and so on). In one embodiment, the reaction tube diameter is greater than or equal to 2 mm but less than or equal to 10 mm. In one embodiment the reaction tube diameter is greater than or equal to 2 mm but less than or equal to 8 mm. In some embodiments, the average internal diameter of the tubular flow line(s) is about 6 mm. Those dimensions provide a particularly advantageous combination of effective mixing of the reaction mixture and specific surface area for effective thermal control. For example, tubular flow line(s) of any of those sizes are sufficiently large to accommodate a static mixer of the kind described herein, yet provide an adequately large specific surface area for effective thermal control. As a result, the reactor can be operated to provide particularly high yields of halogenated alkoxyethane. The resulting reactor represents therefore an advantageous platform for scaled-up production of pharmaceutical grade halogenated alkoxyethane.

In some embodiments, the flow reactor comprises one or more tubular flow line(s) having an internal diameter of no more than about 2 mm, for example of no more than about 1 mm, no more than about 0.5 mm, or no more than 0.1 mm.

A particular advantage offered by such flow reactors is their high surface area to volume ratio, which can range from about 1,000 to well over 20,000 m²/m³. This contrasts significantly with the surface area to volume ratio provided by conventional batch reactors, which is usually in the order of hundreds of m²/m³. As a result of the high surface area to volume ratio, such flow reactors offer excellent heat transfer across the flow line wall, allowing for efficient and fast cooling of exothermic reactions and quasi-isothermal process control.

Accordingly, one or more tubular flow line(s) may be dimensioned to provide a specific surface area (m²/m³) in the range of 100 to 40,000 m²/m³, 200 to 30,000 m²/m³, 300 to 20,000 m²/m³, 500 to 15,000 m²/m³, or 12,000 to 10,000 m²/m³. In some embodiments, the specific surface area is at least 100 m²/m³, at least 200 m²/m³, at least 300 m²/m³, at least 400 m²/m³, at least 500 m²/m³, at least 750 m²/m³, at least 1,000 m²/m³, at least 2,000 m²/m³, at least 3,000 m²/m³, at least 4,000 m²/m³, at least 5,000 m²/m³, at least 7,500 m²/m³, at least 10,000 m²/m³, at least 12,500 m²/m³, at least 15,000 m²/m³, at least 17,500 m²/m³, or at least 20,000 m²/m³. It will be appreciated that the specific surface areas can be measured by a number of techniques and assembled in a number of configurations suitable for industrial scale synthesis.

Also, the mixing efficiency of the reaction mixture flowing through the tubular lines is improved when the internal diameter of the tubular flow line(s) is 8 mm or less, for example 6 mm. In those cases, the flow reactor may comprise one or more tubular flow line(s) having and internal diameter ranging from about 0.1 mm to about 8 mm, or about 1 mm to about 6 mm. In yet a further embodiment the flow reactor comprises one or more tubular flow line(s) having an internal diameter of about 6 mm. The dimension may be varied depending on the throughput scale desired. Effective mixing using tubular flow line(s) of those dimensions may be achieved with or without a static mixer of the kind described herein. In some embodiments, the one or more tubular flow line(s) comprise a static mixer located within the line(s).

The one or more tubular flow line(s) may have an internal surface made of a material that is chemically inert to the reaction components, the halogenated alkoxyethane, and any reaction intermediate or by-product. In that regard, the tubular flow line(s) itself may be made of said material, or have an internal lining made of said material. Further, the material the tubular flow line(s) is made or (or internally lined with) should be of suitable strength and structural integrity to withstand the flow rate pressure(s) and volume(s) of fluid passing through it.

Depending on the nature of the compounds flowing through the line(s), metals, alloys and polymers are particularly preferred as the internal surface material of the tubular flow line(s). Examples of suitable materials for use in the tubular flow line(s) therefore include polyethylene, polypropylene, polyvinyl chloride, a fluorocarbon (e.g. Teflon, polytetrafluoroethylene, polyvinylidene fluoride, fluorinated ethylene propylene, ethylene chlorotrifluoroethylene, polyvinylidene difluoride, a perfluoroalkoxy alkane, etc.), polyether ether ketone, polyethylene, fiberglass-reinforced plastic, Ni-based alloy, and No—Mo-based alloy. The skilled person would be readily capable to identify other materials suitable for use as the internal surface material of the tubular flow line(s) in the invention.

The process of the invention comprises a step of introducing in the flow reactor reaction components comprising (i) a compound of general formula XClC═CF₂, (ii) a base, and (iii) a C₁₋₄ alkanol.

The compound of general formula XClC═CF₂ may be any compound of that formula in which X is —Cl or —F. In some embodiments, X is —Cl, in which case the compound of general formula XClC═CF₂ is Cl₂C═CF₂. In some embodiments, X is —F, in which case the compound of general formula XClC═CF₂ is FCIC═CF₂.

The C₁₋₄ alkanol may be any C₁₋₄ alkanol that promotes addition reaction to the C═C bond of the compound of general formula XClC═CF₂, resulting in a C₁₋₄ alkoxy group bonded on the second carbon. In some embodiments, the C₁₋₄ alkanol is selected from methanol (CH₃OH), ethanol (CH₃CH₂OH), 1-propanol (CH₃CH₂CH₂OH), 2-propanol ((CH₃)₂CHOH), 1-butanol (CH₃CH₂CH₂CH₂OH), 2-butanol (CH₃CH₂CHOHCH₃), 2-methyl-1-propanol ((CH₃)₂CHCH₂OH), 2-methyl-2-propanol ((CH₃)₃COH), and a combination thereof. In some embodiments, the C₁₋₄ alkanol is methanol.

The base may be any base that can catalyse the addition reaction of the C₁₋₄ alkanol to the compound of general formula XClC═CF₂ under the conditions described herein.

In some embodiments, the base comprises an alkali metal base cation. For example, the base may be selected from the group consisting of an alkali metal (e.g. Li, Na and K), an alkali metal salt (e.g. carbonates, acetates and cyanides), an alkali metal hydroxide, an alkali metal alkoxide (e.g. methylate, ethylate, phenolate), and a combination thereof. For example, the base may be selected from sodium methoxide, and potassium methoxide. In some embodiments, the base is an alkali metal hydroxide of general formula M-OH, wherein M is an alkali metal selected from the group consisting of Li, Na and K. In some embodiments, the alkali metal hydroxide is NaOH or KOH. In some embodiments, the base is KOH.

In some embodiments, the base comprises an ammonium or phosphonium base cation. Examples of suitable such bases include tetrabutylammonium hydroxide, benzyl(trimethyl)ammonium hydroxide, N-methyl-N,N,N-trioctylammonium chloride (Aliquat 336), tetraethylammonium hydroxide, tetramethylammonium hydroxide, and tetramethylphosphonium hydroxide.

During formation of the halogenated alkoxyethane, salt intermediates may precipitate within the tubular flow line(s). In those instances, precipitation of the intermediate salt could lead to undesired blockage of the tubular flow line(s). The line(s) would have to be cleaned, leading to unwanted process interruptions. Examples of salt intermediates which may be expected to precipitate during the reaction include salts of an alkali metal (e.g. sodium salts, potassium salts), or halide salts (e.g. chloride, fluoride salts, such as Na fluoride or K fluoride). In those instances, a number of strategies may be adopted to minimise issues deriving from potential precipitation of salt intermediates.

For example, the base may be selected such that it forms a salt soluble in the alkanol during formation of the halogenated alkoxyethane. This advantageously minimises formation of insoluble precipitates along the tubular flow line(s). As a result, the flow reactor can be operated without interrupting fluid flow through the line(s) for significantly longer times relative to conventional procedures. In addition, line cleaning can be less frequent and less onerous, resulting in significant cost savings. In this context, an intermediate salt would be considered “soluble” in the C₁₋₄ alkanol if the salt does not crystallise and precipitate under the reaction conditions. For example, an intermediate salt may be considered “soluble” in the C₁₋₄ alkanol if its solubility in the C₁₋₄ alkanol is at least 0.5 wt % under the reaction conditions. Suitable examples of bases that can form a salt that is soluble in the alkanol include a base comprising an ammonium or phosphonium base cation, such as one selected from tetrabutylammonium hydroxide, benzyl(trimethyl)ammonium hydroxide, N-methyl-N,N,N-trioctylammonium chloride (Aliquat 336), tetraethylammonium hydroxide, tetramethylammonium hydroxide, and tetramethylphosphonium hydroxide.

For example, when the compound of general formula XClC═CF₂ is Cl₂C═CF₂, the base may be selected from tetrabutylammonium hydroxide, benzyl(trimethyl)ammonium hydroxide, N-methyl-N,N,N-trioctylammonium chloride, tetraethylammonium hydroxide, tetramethylammonium hydroxide, and tetramethylphosphonium hydroxide. In those instances, formation and precipitation of salt intermediates can be minimised.

Another strategy to minimise issues deriving from potential precipitation of salt intermediates can be that of adopting a curved arrangement of the one or more tubular flow line(s), for example a coiled arrangement. Accordingly, in some embodiments, the one or more tubular flow line(s) are provided in a coiled arrangement. Examples of suitable coil arrangements are shown in the schematics of FIGS. 2-3. A coiled arrangement may minimise portions of the line(s) within which a precipitate can accumulate, resulting in a more streamlined flow. Also, flow in a coiled arrangement can be more turbulent relative to flow in a rectilinear line. As a result, the extent of mixing is improved resulting in formation of smaller amounts of insoluble salt. As a result, the adoption of tubular flow line(s) in a coiled arrangement can advantageously eliminate the need for static mixers inside the line(s), further reducing accumulation locations for salt precipitates. As a further advantage, having the flow line(s) in a coiled arrangement provides for a more compact reactor, as well as facilitate thermal control of the line(s) through smaller thermal control systems.

In some embodiments, the compound of general formula XClC═CF₂ is Cl₂C═CF₂ and the C₁₋₄ alkanol is methanol. In those instances, the process of the invention allows for the efficient and scalable production of halogenated alkoxyethane compounds such as methoxyflurane (Cl₂HC—CF₂OCH₃). This is particularly advantageous since methoxyflurane is the active ingredient of Penthrox®, which is an effective and rapid-onset short-term analgesic for the initial management of acute trauma pain and brief painful procedures such as wound dressing. Penthrox® is an analgesic used by medical practitioners, the defence forces, ambulance paramedics, sports clubs and surf lifesavers to administer emergency pain relief through inhaler devices known as “Green Whistles”.

Penthrox® has received Regulatory Approvals in a number of major jurisdictions around the world, and is expected to be ubiquitously available as disposable, single-use inhaler devices allowing patients (including children) to self-administer the drug under supervision. Current testing is being performed on advanced inhalers for the self-administration of Penthrox® to be marketed in addition to the Green Whistles. The test inhalers have been developed to be fully integrated pain release systems delivering about 3 ml of Penthrox® to patients in a quick and easy manner. The test inhaler comprises a lock out tab, a plunger that activates the inhaler, and a mouthpiece though which the user can inhale the active Penthrox® composition by normal breathing. Once the lock out tab is removed, the inhaler can be activated by pushing down the plunger. The inhaler would then be set to release the active ingredient through the mouthpiece by the user simply inhaling.

Penthrox® is aimed at becoming available worldwide in facilities that (i) can provide first-aid and emergency services (e.g. hospital emergency, ambulance services, life-saving clubs, etc.), (ii) necessitate mobile, agile, and point-of-care first-aid and emergency services (e.g. the military), and (iii) can market Penthrox® to the general public (e.g. pharmacies) as a mainstream analgesic of choice.

In some embodiments, the compound of general formula XClC═CF₂ is FClC═CF₂ and the C₁₋₄ alkanol is methanol. In those instances, the process of the invention affords efficient and scalable production of ClFHC—CF₂OCH₃ (2-chloro-1,1,2-trifluoroethylmethyl ether). The possibility to produce highly pure and high amounts of ClFHC—CF₂OCH₃ can be particularly advantageous, since that compound is a known precursor in the synthesis of the inhalant anaesthetic enflurane (2-chloro-1,1,2,-trifluoroethyl-difluoromethyl ether). In accordance to a reaction procedure postulated in Scheme 1 below, enflurane (b) can be synthesised by chlorinating ClFHC—CF₂OCH₃ in light (e.g. UV) to give 2-chloro-1,1,2-trifluoroethyldichloromethyl ether (a), followed by substitution of chlorine atoms by fluorine on the dichloromethyl group. The latter is achieved by using, for example, hydrogen fluoride in the presence of antimony(III) chloride, or antimony(III) fluoride with antimony(V) chloride.

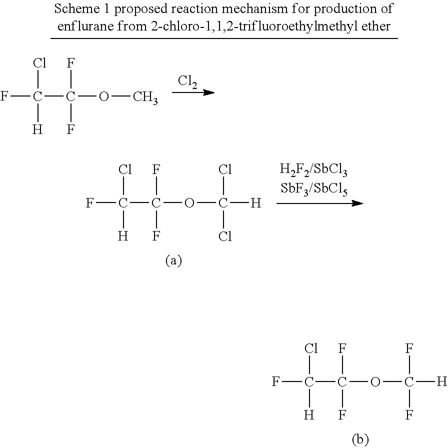

In the method of the invention, the base may be used in any amount conducive to the formation of the halogenated alkoxyethane. In a typical procedure, the base is used in a catalytic amount relative to the compound of general formula XClC═CF₂. By being used in a “catalytic amount”, the base is used in a substoichiometric amount relative to the compound of general formula XClC═CF₂. In some embodiments, the base to XClC═CF₂ compound molar ratio is any decimal fraction of 1. For example, the base to XClC═CF₂compound molar ratio may be about 0.1, about 0.15, about 0.25, about 0.3, about 0.4, about 0.5, about 0.6, about 0.7, about 0.8, or about 0.9.

In some embodiments, the base is used in solution with the C₁₋₄ alkanol. In those instances, the base/alkanol solution may contain the base in an amount between 1% and 30% by weight relative to the total weight of base and C₁₋₄ alkanol. For example, the base may be used in an amount of between about 1% and about 15% by weight, between about 1% and about 15% by weight, or between about 1% and about 5% by weight, relative to the total weight of base and C₁₋₄ alkanol. In some embodiments, the base is used in an amount of about 2% by weight relative to the total weight of base and C₁₋₄ alkanol. In some embodiments, the base is used in an amount of about 5% by weight relative to the total weight of base and C₁₋₄ alkanol. In some embodiments, the base is used in an amount of about 25% by weight relative to the total weight of base and C₁₋₄ alkanol.

In the process of the invention, the reaction components flow through the one or more tubular flow line(s) as a reaction mixture. Typically, each reactor component will be provided as a separate component, and the components mixed to form in a reaction mixture. Mixing of the components may be achieved according to any sequence or means suitable to ensure that the components flow through the one or more tubular flow line(s) as a reaction mixture. For example, each component may be provided in corresponding separate reservoirs, from which they are extracted (e.g. pumped) and mixed with the other components to form the reaction mixture. Said mixing may be performed according to any suitable mixing sequence.

In some embodiments, the base and the C₁₋₄ alkanol are provided as a solution of the kind described herein in a first reservoir, and the XClC═CF₂ compound in a second reservoir. In those instances, the reaction mixture is therefore obtained by mixing (i) the solution of the base and the C₁₋₄ alkanol extracted from the first reservoir with (ii) the compound of general formula XClC═CF₂ extracted from the second reservoir. The mixture is subsequently made to flow (e.g. pumped) through the one or more tubular flow line(s). Examples of such arrangements are shown in the schematics of FIGS. 1 and 3, upstream of reactor (2) and the tubular flow line, respectively.

The base/alkanol solution and the XClC═CF₂ compound may be mixed to form the reaction mixture by any means known to the skilled person.

In some instances, the base/alkanol solution and the XClC═CF₂ compound are mixed by simply flowing them through lines that interject to form a single flow line, for example in a T- or Y- configuration. In those cases, the resulting single flow line may be the feed of the one or more tubular flow line(s) of the flow reactor.

In other configurations, one of the base/alkanol solution line and the XClC═CF₂ compound line is fed into the one or more tubular flow line(s) of the flow reactor, and the other of the base/alkanol solution and the XClC═CF₂ compound is fed into the tubular flow line(s) through an interjecting line. As a result, the base/alkanol solution and the XClC═CF₂ compound mix to form the reaction mixture. An example of such configuration is shown in FIG. 2, described in more detail further below.

In yet further configurations, the base/alkanol solution and the XClC═CF₂ compound are mixed in a mixing unit located upstream of the one or more tubular flow line(s). This can advantageously ensure a high degree of mixing between all reaction components before they enter into the tubular flow line(s) as a reaction mixture. As a result, fast formation of highly pure alogenated alkoxyethane can be achieved, even in the absence of static mixers within the flow line(s).

The mixing unit may or may not be an integral component of the flow reactor. The mixing unit may be an active mixing unit, in which mixing is achieved by providing external energy. Examples of such units suitable for use in the process of the invention include units that impart time-pulsing flow owing to a periodical change of pumping energy or electrical fields, acoustic fluid shaking, ultrasound, electrowetting-based droplet shaking, micro-stirrers, and the like. In alternative configurations, the mixing unit may be a passive mixing unit, in which mixing is achieved by combining the base/alkanol solution line and the XClC═CF₂ compound line into one single line. Examples of such units suitable for use in the process of the invention include Y- and T-type flow junctions, multi-laminating mixers, split-and-recombine mixers, chaotic mixers, jet colliding mixers, recirculation flow-mixers, and the like. Typical design for passive mixing units include T- and Y-flow configurations, interdigital- and bifurcation flow distribution structures, focusing structures for flow compression, repeated flow division- and recombination structures, flow obstacles within the line, meander-like or zig-zag channels, multi-hole plates, tiny nozzles, and the like. A schematic of an arrangement involving the use of a mixing unit located upstream of the one or more tubular flow line(s) is shown in FIG. 3.

In some embodiments, the one or more tubular flow line(s) comprise an inline static mixer. This is particularly advantageous as the internal cross-sectional area of the flow line(s) increases (for example above 5 mm²). In those instances, diffusion-driven intermixing of the components as they flow through the line (which can be a major driver of mixing in tubular flow line(s) of small internal cross-sectional area) may not be sufficient to promote intimate mixing. A static mixer within the tubular flow line(s) can therefore be implemented to induce multi-lamellation of the flowing fluid or the formation of vortices within the volume of the flowing fluid, thereby increasing mixing efficiency.

Examples of suitable static mixers include baffles, helical mixers, spinning disks, and spinning tubes. As the skilled person will appreciate, the static mixer may be made of any material that is chemically inert to the reaction components, the halogenated alkoxyethane, and any reaction by-product and/or intermediate. Examples of suitable materials in that regard include polyethylene, polypropylene, polyvinyl chloride, a fluorocarbon (e.g. Teflon, polytetrafluoroethylene, polyvinylidene fluoride, fluorinated ethylene propylene, ethylene chlorotrifluoroethylene, polyvinylidene difluoride, a perfluoroalkoxy alkane, etc.), polyether ether ketone, polyethylene, fiberglass-reinforced plastic, Ni-based alloy, and No—Mo-based alloy. The skilled person would be readily capable to identify other materials suitable for use in the static mixer.

While the above discussion is made in the context of materials used to make (or internally line) the one or more tubular flow line(s), it will be understood that similar considerations apply to the material used to make (or internally line/coat) any element (or part thereof) of the system/apparatus used to perform the process and that is expected to come into contact with any one of the reaction components, product, intermediate, by-product(s), and/or mixture thereof. That is, it will be understood that any element (or part thereof) of the system/apparatus used to perform the process that is expected to come into contact with any one of the reaction components, product, intermediate, by-product(s), and/or mixture thereof would have to be made of a material that is chemically inert to said reaction component, product, intermediate, by-product(s) (which may include strong acids such as HCl or HF), and/or mixture thereof. Accordingly, any such element(s) may be made (or lined with, as appropriate) by a material of the kind described herein.

For example, any reservoir that is part of the system/apparatus used to perform the process may be made of (or internally lined with) a material that is chemically inert to the chemical component or mixture the reservoir is intended to store. Similarly, relevant components of pumps that may be used to pump a reaction component, product, intermediate, by-product(s), and/or any mixture thereof may be made of a material that is chemically inert to said reaction component, product, intermediate, by-product(s), and/or mixture thereof. Also, relevant components of mixing units of the kind described herein which may come into contact with a reaction component, product, intermediate, by-product(s), and/or any mixture thereof may be made of a material that is chemically inert to said reaction component, product, by-product(s), and/or mixture thereof. Examples of suitable materials in that regard include polyethylene, polypropylene, polyvinyl chloride, a fluorocarbon (e.g. Teflon, polytetrafluoroethylene, polyvinylidene fluoride, fluorinated ethylene propylene, ethylene chlorotrifluoroethylene, polyvinylidene difluoride, a perfluoroalkoxy alkane, etc.), polyether ether ketone, polyethylene, fiberglass-reinforced plastic, Ni-based alloy, and No—Mo-based alloy. The skilled person would be readily capable to identify other materials suitable for use in any of the components of the reactor to ensure safe handling of all mixtures and compounds involved in the invention.

In the process of the invention, the relative amount of the reaction components in the reaction mixture can be modulated by tuning the flow rate of each component when it is mixed with the others. For example, the relative amount of the reaction components in the reaction mixture can be modulated by tuning the flow rate of the base/alkanol solution line relative to that of the XClC═CF₂ compound line. The ratio between the flow rate of the base/alkanol solution line relative to that of the compound of general formula XClC═CF₂ line may be any ratio that is conducive to the formation of the halogenated alkoxyethane. For example, the reaction mixture may be obtained by combining (i) a solution of the C₁₋₄ alkanol and the base with (ii) the compound of general formula XClC═CF₂ according to a flow rate ratio from 1:1 to 10:1. In some embodiments, said flow rate ratio is between 1:1 to 6:1, from 2:1 to 6:1, from 3:1 to 6:1, or from 4:1 to 5:1.

In that context, each of the base/alkanol solution line and the XClC═CF₂ compound line may be operated at a flow rate that is conducive to the formation of the halogenated alkoxyethane upon mixing of the base/alkanol solution with the XClC═CF₂ compound. In one embodiment, the flow rate of each individual line is at least 1 ml/min. For example, the flow rate of each individual line may be at least about 5 ml/min, at least about 25 ml/min, at least about 50 ml/min, at least about 100 ml/min, at least about 200 ml/min, at least about 500 ml/min, at least about 1,000 ml/min, at least about 1,500 ml/min, or at least about 2,000 ml/min. In some embodiments, the flow rate of each individual line is about 250 ml/min.

In some embodiments, the base/alkanol solution is pumped or otherwise supplied into the mixer unit or the one or more tubular flow line(s) at a flow rate greater than 5 ml/min but less than 2,000 ml/min, and the XClC═CF₂ compound is pumped or otherwise supplied into the mixer unit or the one or more tubular flow line(s) at a flow rate greater than 5 ml/min but less than 2,000 ml/min. In one embodiment, the base/alkanol solution is pumped or otherwise supplied into the mixer unit or the one or more tubular flow line(s) at a flow rate greater than or equal to 50 ml/min but less than or equal to 500 ml/min, and the XClC═CF₂ compound is pumped or otherwise supplied into the mixer unit or the one or more tubular flow line(s) at a flow rate greater than or equal to 50 ml/min but less than or equal to 500 ml/min. In one embodiment, the base/alkanol solution is pumped or otherwise supplied into the mixer unit or the one or more tubular flow line(s) at a flow rate of about 250 ml/min, and the XClC═CF₂ compound is pumped or otherwise supplied into the mixer unit or the one or more tubular flow line(s) at a flow rate of about 50 ml/min.

In the process of the invention, the reaction mixture may flow through the one or more tubular flow line(s) at any flow rate that is conducive to generation of the halogenated alkoxyethane. In some embodiments, the reaction mixture flows through the one or more tubular flow line(s) at a flow rate of at least about 1 ml/min. For example, the reaction mixture may flow through the one or more tubular flow line(s) at a flow rate of at least about 5 ml/min, at least about 25 ml/min, at least about 50 ml/min, at least about 100 ml/min, at least about 250 ml/min, at least about 500 ml/min, at least about 750 ml/min, at least about 1 L/min, or at least about 2 L/min.

The one or more tubular flow line(s) may provide for any internal volume conducive to generation of the halogenated alkoxyethane. For avoidance of doubt, by “internal volume” of the one or more tubular flow line(s) is meant the volume of the internal cavity of the tubular flow line(s) through which the reaction components flow as a reaction mixture. In other words, the “internal volume” of the one or more tubular flow line(s) corresponds to the total volume of fluid present in the tubular flow line(s) at any given time, when the reactor is in operation.

In one embodiment, the one or more tubular flow line(s) has/have a total internal volume of at least 100 mL, at least 250 mL, at least 500 mL, at least 750 mL, at least 1 L, at least 1.5 L, at least 2 L. For example, the one or more tubular flow line(s) may have a total internal volume in the range of 100 ml to 2 L, for example less than or equal to 1 L (and including any integer there between, and/or fraction thereof, for example, 100 ml, 100.1 ml, etc.). In one embodiment the one or more tubular flow line(s) has/have a total internal volume greater than or equal to 200 ml but less than or equal to 600 ml. For example, the one or more tubular flow line(s) may have a total internal volume greater than or equal to 250 ml but less than or equal to 500 ml. In one embodiment, the one or more tubular flow line(s) has/have a total internal volume greater than or equal to 200 ml but less than or equal to 350 ml.

The one or more tubular flow line(s) may be of any length allowing for generation of the halogenated alkoxyethane. The length of the tubular flow line(s) may be selected depending on the internal cross-sectional area and the desired volume for the reaction. In one embodiment the tubular flow line(s) has/have a length greater than or equal to 1 metre but less than or equal to 50 meters (and including any integer there between, and/or fraction thereof, for example, 1 meter, 1.1 meter, 1.15 meter, 1.2 meter, 1.25 meter, and so on). In one embodiment the tubular flow line(s) has/have a length greater than or equal to 5 meters but less than or equal to 25 meters. In another embodiment the tubular flow line(s) has/have a length greater than or equal to 10 meters but less than or equal to 25 meters. In some embodiments, the tubular flow line(s) has/have a length of at least 0.5 meters, at least 1 meter, at least 5 meters, at least 10 meters, or at least 25 meters.

The volumetric residence time of fluid flowing through the one or more tubular flow line(s) can be determined by the ratio of the total internal volume of the tubular flow line(s) to the flow rate of the fluid flowing through the tubular flow line(s). In turn, the latter may be determined by the sum of the flow rate of all reagent component lines converging into the one or more tubular flow line(s). In the process of the invention, the flow reactor may be operated to obtain any residence time of fluid flowing through the one or more tubular flow line(s) that is conducive to generation of the halogenated alkoxyethane. For example, the flow reactor may be operated to provide a residence time of less than about 250 minutes. In some embodiments, the flow reactor is operated to provide a residence time of less than about 200 minutes, less than about 100 minutes, less than about 50 minutes, less than about 25 minutes, less than about 20 minutes, less than about 15 minutes, less than about 10 minutes, less than about 5 minutes, less than about 2.5 minutes, less than about 2 minutes, or less than about 1 minute. In some embodiments, the flow reactor is operated to provide a residence time of about 1 minute.

The flow reactor in the process of the invention may be operated at any pressure conducive to generation of the halogenated alkoxyethane. For example, in the process of the invention the reaction components may flow through the one or more tubular flow line(s) at a pressure of at least 15 bar. For avoidance of doubt, values of pressure used herein refer to gauge pressure.

In that context, the procedure of the invention advantageously allows for production of halogenated alkoxyethane at significantly lower pressure than conventional procedures. For example, in the process of the invention, the reaction components may flow through the one or more tubular flow line(s) at a pressure of less than 30 bar. In some embodiments, the reaction components flow through the one or more tubular flow line(s) at a pressure of less than 20 bar, less than 15 bar, or less than 10 bar. In some embodiments, the reaction components flow through the one or more tubular flow line(s) at a pressure of 10-15 bar. In some embodiments, the reaction components flow through the one or more tubular flow line(s) at a pressure of about 18 bar.

In the process of the invention, the halogenated alkoxyethane forms at least upon the reaction components mixing. The reaction is exothermic and reaction heat can be continuously extracted by any means known to the skilled person. Suitable heat control strategies include the provision of a cooling jacket, a heat exchanger, or a combination thereof in thermal contact with at least a portion of the one or more tubular flow line(s). The jacket can maintain the temperature of the fluid flowing within the flow line(s) by way of a cooling medium provided to the jacket by a cooling line. The cooling medium may be any medium known to the skilled person, for example water, glycol, or a water/glycol mixture. The cooling medium or cooling jacket or heat exchanger may form part of an external casing within which the flow line(s) are located.

In some embodiments, the halogenated alkoxyethane is also formed by cooling the reaction mixture to a temperature of down to −15° C. For example, the reaction mixture may be cooled to a temperature of down to about −10° C., down to about −5° C., down to about −2.5° C., down to about −1° C., down to about 0° C., down to about 5° C., or down to about −10° C. In some embodiments, the halogenated alkoxyethane is formed at a temperature from 0° C. to 5° C.

The temperature of any of the reagent compounds may also be controlled to a desired value. For instance, the base/alkanol solution may be used at room temperature. In some embodiments, the base/alkanol solution is used at a temperature below 10° C., for example below 5° C., or between 0° C. and 5° C. In some embodiments, the XClC═CF₂ compound is used at room temperature. In some embodiments, the XClC═CF₂ compound is used at a temperature below 10° C., for example below 5° C., or between 0° C. and 5° C. Accordingly, in some embodiments one or more reagent compound(s) are cooled prior to being mixed to form the reaction mixture, such that the one or more reagent compound(s) is/are in liquid form when the reaction mixture forms. Cooling any of the reagent components may be necessary to ensure they are used in liquid form in the flow reactor. This may be achieved by any means known to the skilled person. For example, reservoirs of either or both the base/alkanol solution and the XClC═CF₂ compound may be temperature controlled. In some embodiments, either or both the base/alkanol solution and the XClC═CF₂ compound are provided in corresponding temperature controlled reservoir. Such temperature control may be achieved by cooling strategies of the kind described herein (e.g. cooling jacket, a heat exchanger, or a combination thereof). Alternatively, or at the same time, cooling of one or more reagent component(s) may be achieved by a temperature controlled reservoir pump, for example a pump provided with a cooling system of the kind described herein (e.g. cooling jacket, a heat exchanger, or a combination thereof).

As used herein, “room temperature” refers to ambient temperatures which may be, for example, between 10° C. to 40° C. but is more typically between 15° C. to 30° C. For example, room temperature may be a temperature between 20° C. and 25° C.

In the process of the invention, the halogenated alkoxyethane flows out of the flow reactor in a reactor effluent. This may be achieved by any means known to the skilled person. When the flow reactor comprises two or more tubular flow lines, the lines would typically converge to form a single outlet from which the effluent exits the reactor. The effluent may exit the reactor at a flow rate that depends on the operational parameters of the reactor. For example, the reactor effluent containing the halogenated alkoxyethane may exit the reactor at a flow rate of at least 5 ml/min. In some embodiments, the reactor effluent containing the halogenated alkoxyethane exits the reactor at a flow rate of at least 10 ml/min, at least 25 ml/min, at least 50 ml/min, at least 100 ml/min, at least 250 ml/min, at least 500 ml/min, at least 750 ml/min, at least 1 L/min, at least 1.5 L/min, or at least 2 L/min.

The effluent may contain an amount of halogenated alkoxyethane that is dependent on the operational parameters of the reactor. In some embodiments, the reactor effluent contains at least 70% by volume, at least 80% by volume, at least 90% by volume, or at least 95% by volume of the halogenated alkoxyethane. Advantageously, the process of the invention affords higher conversion yields than conventional procedures. Accordingly, in some embodiments the reactor effluent contains at least 90% by volume of the halogenated alkoxyethane.

In some embodiments, the process also comprises a step of mixing the reactor effluent with a polar solvent. For example, the process may comprise a step of mixing the reactor effluent with water. This may provide a biphasic mixture that can be used in the context of the purification procedure described herein. The polar solvent (e.g. water) may be mixed with the reactor effluent by any of the mixing procedures described herein. For example, one or more lines carrying the polar solvent (e.g. water) from a reservoir may be made to interject the reactor effluent line, and the polar solvent made to flow (e.g. pumped) from a dedicated reservoir. Alternatively, the polar solvent (e.g. water) may be mixed with the reactor effluent by way of a mixing unit of the kind described herein.

The polar solvent (e.g. water) may be provided according to any flow rate that is suitable to obtain a biphasic mixture with the reactor effluent. In some embodiments, the polar solvent (e.g. water) is provided according to a flow rate of at least 5 ml/min, at least 25 ml/min, at least 50 ml/min, at least 100 ml/min, at least 250 ml/min, at least 500 ml/min, at least 750 ml/min, at least 1,000 ml/min, at least 1,500 ml/min, at least 2,000 ml/min, at least 2,500 ml/min, at least 3,000 ml/min, at least 4,000 ml/min, or at least 5,000 ml/min. In some embodiments, the polar solvent (e.g. water) is provided according to a flow rate greater than or equal to 5 ml/min but less than or equal to 5,000 ml/min. For example, the polar solvent (e.g. water) may be provided according to a flow rate selected from an integer in the range 5 ml/min to 500 ml/min, for example in the range 25 ml/min to 250 ml/min. In some embodiments, the polar solvent (e.g. water) is provided according to a flow rate of 250 ml/min. In some embodiment, the polar solvent (e.g. water) may be provided at any pressure that is suitable for effective mixing with the reactor effluent. For example, the polar solvent (e.g. water) may be pumped at a pressure of >15 bar. In some instances, the polar solvent (e.g. water) may also be pumped at a pressure of <15 bar. In some embodiments, the polar solvent (e.g. water) may be pumped at a pressure of 10-15 bar. Typically, the polar solvent (e.g. water) may be pumped at room temperature.

The reactor effluent may also contain additional compounds present in the effluent as impurities. Depending on the reactor conditions and/or the nature of the reaction components, said impurities may comprise one or more reaction by-product(s) and/or one or more unreacted reaction components. The nature of the impurities depends on the reaction conditions and/or the nature of the reaction components. For example, when the process of the invention is performed to produce methoxyflurane, the impurities may comprise methanol, dichloro-difluoroethylene (DCDFE), halomar (2-chloro-1,1,2-trifluoroethyl methyl ether), chloroform and/or 1,1-dichloro-2-fluoro-2-methoxyethene (vinyl ether). In one such embodiments, the impurities comprise 1,1-dichloro-2-fluoro-2-methoxyethene.

Depending on the reactor conditions and/or the nature of the reaction components, said impurities may also be present in an amount that can range from less than 5% up to about 30% by volume of the effluent. Advantageously, the process of the invention can ensure that the halogenated alkoxyethane can be produced at a significantly higher purity (i.e. above 90% by volume of effluent) relative to conventional synthesis procedures. In some embodiments, the reactor effluent contains less than 5% impurities by volume.

If necessary, as part of the process of the invention, the halogenated alkoxyethane exiting the flow reactor in the effluent may be subject to purification. These may conveniently be achieved by subjecting the reactor effluent to an in-line purification technique (i.e. whereby the purification technique is integrated into the process).

Accordingly, in some embodiments the process of the invention further comprises a purification procedure that comprises the steps of a) mixing the reactor effluent with a polar solvent to induce phase separation between a polar phase and an organic phase comprising the halogenated alkoxyethane, b) separating the organic phase from the polar phase, c) treating the organic phase with an amine, d) washing the organic phase with an acid solution, e) desiccating the organic phase, and f) distilling the organic phase to obtain a purified distillate comprising the halogenated alkoxyethane.

The purification procedure may be in-line, in which case the reactor effluent is subject to the purification procedure downstream of the flow reactor. By including an in-line purification procedure, the process of the invention advantageously provides for the continuous production of purified halogenated alkoxyethane. Alternatively, the purification procedure may be performed separately, in which case the reactor effluent would be initially collected upon exiting the floor reactor with no further processing, optionally stored, and subsequently purified.

The polar solvent used in step a) of the purification procedure may be any polar solvent that can induce phase separation of the effluent between a polar phase and an organic phase that comprises the halogenated alkoxyethane. For example, the polar solvent may be water. A skilled person would be capable to identify other polar solvents suitable for this purpose.

Separation of the organic phase from the polar phase may be effected according to any means known to the skilled person. For example, separation of the organic phase from the polar phase may be effected by way of a gravity separator (e.g. a phase separation tank), a super-hydrophobic mesh, a super-oleophobic mesh, and the like. A skilled person would be capable to identify suitable means and procedures for the effective separation of the organic phase from the polar phase for the purpose of this step.

In step c) of the purification procedure, the organic phase comprising the halogenated alkoxyethane is treated with an amine. Typically, the organic phase would be treated with excess amine. Depending on the nature of the impurities, the amine may be a primary or a secondary amine. Examples of suitable amines for this purpose include ethylenediamine (1,2-diamnoethane), 1,3-diaminopropane, diethylenetriamine, di-n-propylamine, n-butylamine, ethanolamine, pyrrolidine, 2-aminobutane, and a mixture thereof. In some embodiments, the amine is selected from ethylenediamine, 1,3-diaminopropane, diethylenetriamine, and a mixture thereof.

In step d) of the purification procedure, following treatment with amine the organic phase is washed with an acid solution. This ensures effective removal of impurities such as vinyl ether, which would typically form when the process is used to produce methoxyflurane. The acid solution may be an aqueous acid solution. The acid used in the acid solution may be any acid effective to remove impurities such as vinyl ether. Examples of suitable acids for use in the purification procedure include hydrochloric acid, sulfuric acid, sulphurous acid, methanesulfonic acid, trifluoromethanesulfonic acid, phosphoric acid, acetic acid, trifluoroacetic acid, nitric acid, nitrous acid, hypochlorous acid, chlorous acid, chloric acid, perchloric acid, and a combination thereof. In one embodiment, the acid is methanesulfonic acid (MSA). In some embodiments, the acid solution is at least a 10%, at least a 20%, at least at 30%, or at least a 40% acid solution. The organic phase may be washed with any effective amount of acid solution. For example, the acid solution may be added to the organic phase according to a 0.25:1, 0.5:1, 1:1, or 2:1 volume ratio (acid solution to organic phase).

The acid treatment of the organic phase in the purification procedure has surprisingly be found effective for obtaining pharmaceutical grade halogenated alkoxyethane (e.g. 99.9%). In that regard, the acid is particularly effective for efficient removal of impurities while remaining inert towards the halogenated alkoxyethane. For example, in the purification procedure for obtaining pharmaceutical grade methoxyflurane the use of an acid of the kind described herein can be particularly effective for the selective removal of vinyl ether impurities (e.g. 1,1-dichloro-2-fluoro-2-methoxyethene) while retaining methoxyflurane formed in the reaction. This has been found to be particularly advantageous for the synthesis of methoxyflurane with purity above 99%, for example up to 99.9% purity. In that regard, methanesulfonic acid has been found to be particularly effective.

Based on the above, it is therefore believed that the treatment of the reactor effluent (or an organic phase thereof) with an acid may be unique and advantageous on its own right. Accordingly, in some embodiments the process of the invention further comprises a purification step in which the reactor effluent, or an organic phase derived from the reactor effluent and containing the halogenated alkoxyethane, is treated with an acid. Said organic phase may be for example the organic phase resulting from mixing the reactor effluent with a polar solvent (e.g. water) downstream of the tubular flow line(s). The acid may be an acid of the kind described herein. For example, when the process of the invention further comprises a purification step in which the reactor effluent, or an organic phase derived from the reactor effluent and containing the halogenated alkoxyethane, is treated with an acid, said acid may be selected from hydrochloric acid, sulfuric acid, sulphurous acid, methanesulfonic acid, trifluoromethanesulfonic acid, phosphoric acid, acetic acid, trifluoroacetic acid, nitric acid, nitrous acid, hypochlorous acid, chlorous acid, chloric acid, perchloric acid, and a combination thereof. In some embodiments, the acid is methanesulfonic acid. In these instances, the purification step may be performed at any of the purification conditions described herein. For instance, when the process of the invention further comprises a purification step in which the reactor effluent, or an organic phase derived from the reactor effluent and containing the halogenated alkoxyethane, is treated with an acid, the acid is provided as an acid solution (e.g. an acid aqueous solution) which is at least a 10%, at least a 20%, at least at 30%, or at least a 40% acid solution. In these instances, the organic phase may be washed with any effective amount of acid solution. For example, the acid solution may be added to the portion of the reactor effluent containing the halogenated alkoxyethane according to a 0.25:1, 0.5:1, 1:1, or 2:1 volume ratio (acid solution to portion of the reactor effluent containing the halogenated alkoxyethane). Advantageously, when the process of the invention further comprises a purification step in which the reactor effluent, or an organic phase derived from the reactor effluent and containing the halogenated alkoxyethane, is treated with an acid, the halogenated alkoxyethane can be obtained at a purity above 99%, for example up to 99.9% purity.

Desiccation of the so formed organic phase in step c) of the purification procedure may be effected according to any means known to the skilled person.

The purification procedure further includes a step of distilling the desiccated organic phase. This may be effected according to any means known to the skilled person.

In some embodiments, the purification procedure comprises the addition of an oxidizing agent. Said oxidising agent has been found to be effective in the removal of impurities such as vinyl impurities (e.g vinyl ether). The oxidising agent may therefore be used in addition, or as an alternative, to the acid solution. Accordingly, in some embodiments the process of the invention further includes a purification procedure that comprises the steps of a) mixing the reactor effluent with a polar solvent to induce phase separation between a polar phase and an organic phase comprising the halogenated alkoxyethane, b) separating the organic phase from the polar phase, c) treating the organic phase with an amine, d) washing the organic phase with an oxidizing agent, e) desiccating the organic phase, and f) distilling the organic phase to obtain a purified distillate comprising the halogenated alkoxyethane. Any oxidising agent that is effective in the removal of impurities such as vinyl ether may be used in the purification procedure. Examples of suitable oxidizing agents include oxygen, ozone, oxone (with or without water), a peroxide, a hydroperoxide, a hypochlorite (e.g. sodium hypochlorite), and a mixture thereof.

The organic phase may be left to react with the acid solution and/or the oxidising agent for any length of time that is effective to ensure that impurities such as vinyl ether are no longer detectable by gas chromatography. For example, the organic phase may be left to react with the acid solution and/or the oxidising agent for a length of time there is sufficient to reduce purities, such as vinyl ether, to below 0.01% by weight. In some embodiments, the organic phase is left to react with the acid solution and/or the oxidising agent for at least 1, at least 2, at least 5, at least 10, at least 24, at least 48, or at least 72 hours.

An example set up which may be used to perform the process of the invention is shown in the schematics of FIG. 1. Apparatus (1) is made of a flow reactor (2), within which the one or more tubular flow line(s) is/are located (see for example FIG. 2). Apparatus (1) includes reservoir (7), within which the base/alkanol solution (5) can be stored, and reservoir (8), within which the XClC═CF₂ compound (6) can be stored. In the schematics, reservoir (8) is represented in the form of an inverted gas cylinder, which may be used to store gaseous XClC═CF₂ compound (e.g. dichlorodifluoroethylene), if needed. Alternatively, reservoir (8) may be a liquid tank for XClHC—CXF₂ compound that is in liquid form at the temperature it is stored Individual pumps (3) and (4) extract the base/alkanol solution (5) and the XClC═CF₂ compound (6) and pump them into flow reactor (2) through lines (14) and (15), respectively. In the schematics, cooling jackets (9) and (10) are used to cool the reaction components to below room temperature, for example to about 5° C., by means of a cooling medium which can be delivered from cooling medium chiller unit (11) through pump jacket cooling lines (12). In schematics, cooling medium chiller unit (11) is also shown to be connected to the flow reactor (2). Back-pressure system (13) may be operated between an open and closed position to set a desired pressure within the one or more tubular flow line(s).

During operation, the base/alkanol solution (5) and the XClC═CF₂ compound (6) enter flow reactor (2) via inlet ports (19) and (18), respectively. Once inside, they are mixed to form the reaction mixture by having their respective lines interject (as shown in more detail in the schematic of FIG. 2). The reactor effluent containing the halogenated alkoxyethane exits flow reactor (2) via outlet port (20), and may be mixed with water stored in reservoir (17) and pumped by pump (16) through line (21). The mixture of the reactor effluent and water can then flow through static mixer (22), and the mixture collected for further purification from line (23).

FIG. 2 shows a cut-away side view of an exemplary embodiment flow reactor suitable for use in the process of the invention. One of the base/alkanol solution and the XClC═CF₂ compound is fed, for example pumped, at an end of a tubular flow line through inlet (25). The other of the base/alkanol solution and the XClC═CF₂ compound is fed, for example pumped, through inlet (26) into a line that interjects the tubular flow line at a mixing port (MP). As a result, the base/alkanol solution and the XClC═CF₂ compound mix to form the reaction mixture, which flows downstream of the mixing port through the coiled tubular flow line (27). Casing (28) may be filled with a cooling medium (for example chilled water) that can be introduced through port (29). The halogenated alkoxyethane forms along the flow line as the mixture flows through it, and an effluent containing the halogenated alkoxyethane can be collected from outlet (30). The effluent can subsequently be mixed with water provided through line (31) in static mixer (32). The reactor may comprise one or more temperature probes (TP), a mixing port (MP), a pressure transducer (PT) and/or back pressure regulatory (BPR).

Advantageously, the process of the invention can be efficiently scaled-up for commercial-scale production of the halogenated alkoxyethane. This may be achieved, for instance, by increasing the number of tubular flow lines in the flow reactor. Since the internal geometry of each flow line is maintained, and the reaction conditions remain identical within each flow line, the process can be adapted to produce higher amounts halogenated alkoxyethane with minimal re-optimization of the reaction conditions. This advantageously ensures fast and seamless transfer from lab-scale testing to production. Accordingly, in some embodiments the flow reactor comprises at least 5 tubular flow lines, at least 10 tubular flow lines, at least 25 tubular flow lines, at least 50 tubular flow lines, or at least 100 tubular flow lines. Alternatively, or in addition to the above, production may be scaled-up by running a number of flow reactors in parallel. Accordingly, in some embodiments the process comprises a step of introducing reactor components of the kind described herein into at least 2, at least 5, at least 10, at least 20 flow reactors connected in a parallel flow arrangement.

Specific embodiments of the invention will now be described with reference to the following non-limiting examples.

### EXAMPLES

### Example 1

A 2.5% KOH solution in methanol was first prepared by dissolving approximately 1.67 kg of KOH in approximately 66.7 L of methanol (analytical grade). All solids were dissolved, and the dissolution was observed to be exothermic. The following parameters for the KOH/methanol solution were adopted.

KOH/methanol solution volume: 66.67 L

Reservoir: Blue plastic mauser drum

KOH/methanol solution pump flow rate: 250 ml/min

Pump refill rate: 400 ml/min

Cooling Jacket: Yes, connected to water chiller unit.

Dichlorodifluoroethylene (DCDFE) (gas) was used to form an inverse gas bottle (care had to be taken to maintain the system dry, since DCDFE reacts with moisture over time to release extremely corrosive HF). The following parameters for the DCDFE line were adopted.

DCDFE reservoir volume: 13.10 L

Reservoir: Gas cylinder

DCDFE pump flow rate: 50 ml/min

Pump refill rate: 70 ml/min

Cooling Jacket: Yes, connected to water chiller unit.

A water line interjecting the reactor effluent (i.e. downstream of the flow reactor) was used to mix the reactor effluent with water (e.g. potable water) as the reactor effluent exits the flow reactor. The following parameters for the water line were adopted.

Water: Water (potable)

Water reservoir volume: 80.0 L

Water pump flow rate: 250 ml/min

The reaction components were mixed to form a reaction mixture, and made to flow within the flow reactor through tubular flow lines arranged to provide parallel flow within the reactor. Parameters of the flow reactor and respective tubular flow lines were as follows.

Total volume of flow lines: 1 L

Individual tubular flow line diameter (internal): ¼ inch (6.35 mm)

Tubular flow lines arrangement: 5×5 linear array

Total flow rate of reaction mixture: 300 ml/min

Residence time: about 3.33 minutes

Static mixer (located in the flow reactor): Yes

Details of the apparatus and the procedure adopted for the purpose of this example are described below.

1. An example of a suitable apparatus (1) comprising a flow reactor (2) for use in accordance with this Example is illustrated in FIG. 1.

2. Pumps (3) and (4) were primed with methanol (reagent grade) prior to charging/filling them with a KOH/methanol solution (2.5% KOH in methanol) (5) and DCDFE (6) from the first reservoir (7) and second reservoir (inverted gas cylinder) (8), respectively.

3. To conduct the reaction at a desirable temperature the pumps were provided with cooling jackets (9) and (10), which were cooled to below 5° C. by chilled water delivered from water chiller unit (11) via pump jacket cooling lines (12), the chiller unit (11) having been set to between 0° C. to 5° C. Water chiller unit (11) may also be connected to the flow reactor, if necessary.

4. All lines were primed by setting back-pressure system (13) to an open position to provide a pressure of approximately 10 bar (noting that during continuous flow operation the conduit system pressure will run at approximately 12-14 bar).

5. During operation, the KOH/methanol solution (5) was pumped according to a desired flow rate (250 ml/min) from reservoir (7) through to the flow reactor via pump line (14).

6. During operation, DCDFE (6) was pumped continuously at a desired flow rate (50 ml/min) from reservoir (8) through to the flow reactor via pump line (15).

7. Water was pumped continuously with pump (16) at a flow rate of 250 ml/min from reservoir (17).

8. Pumps (3) and (4) were started simultaneously, while pump (16) was delayed for up to 60 seconds.

9. The KOH/methanol solution (2.5% KOH in methanol) (5) and DCDFE (6) entered flow reactor (2) via inlet ports (18) and (19).

10. The composition of the mixture of reactor components and reaction products in the reactor effluent and in a phase separation vessel (not shown) were monitored by gas chromatography and recorded at 20 minute intervals starting at T=10 minutes. The results for the production of the desired ether product methoxyflurane and vinyl impurity (methoxyethene) as sampled during the reaction run time are reproduced in Tables 1 and 2 respectively below.

11. As the reactor effluent exits the flow reactor via outlet port (20) it mixes with water (21) pumped from reservoir (17).

12. After passing through a static mixer (22), the water/effluent mixture was collected through line (23) at a rate of approximately 57.4 ml/min over a total reaction time 270 minutes. A total reaction volume of 15.44 L was collected, which comprised the desired ether product (methoxyflurane) according to an approximately 90% crude yield.

13. The crude methoxyflurane product mixture can, if required, be held for up to a week before purification.

TABLE 1
Composition of reactor effluent

| | RT | Spec | 10 | 30 | 50 | 70 | 90 | 110 | 130 | 150 |
|---|---|---|---|---|---|---|---|---|---|---|
| Peak | (min) | (%) | min | min | min | min | min | min | min | min |
| MeOH | ~2.3 | <5.0 | 0.226 | 1.015 | 0.107 | 0.206 | 0.231 | 0.827 | 0.071 | 0.095 |
| DCDFE | ~2.5 | <0.5 | 1.485 | 1.721 | 1.420 | 1.772 | 1.651 | 1.572 | 1.538 | 1.724 |
| Methoxyflurane | ~4.3 | >95 | 97.734 | 96.713 | 97.653 | 97.473 | 97.577 | 97.055 | 97.851 | 97.642 |
| Methoxyethene | ~4.5 | <1.0 | 0.296 | 0.296 | 0.299 | 0.304 | 0.315 | 0.310 | 0.316 | 0.318 |

where RT = retention time; spec = Specification (as % Total Peak Area i.e. TPA %)

TABLE 2
Composition of organic phase in phase separator

| | RT | Spec | 10 | 30 | 50 | 70 | 90 | 110 | 130 | 150 |
|---|---|---|---|---|---|---|---|---|---|---|
| Peak | (min) | (%) | min | min | min | min | min | min | min | min |
| MeOH | ~2.3 | <5.0 | 0.156 | 0.096 | 0.078 | 0.048 | 0.078 | 0.092 | 0.073 | 0.101 |
| DCDFE | ~2.5 | <0.5 | 1.002 | 1.121 | 1.257 | 1.252 | 1.218 | 1.274 | 1.319 | 0.887 |
| Methoxyflurane | ~4.3 | >95 | 98.278 | 98.223 | 98.100 | 98.144 | 98.141 | 98.077 | 98.028 | 98.482 |
| Methoxyethene | ~4.5 | <1.0 | 0.230 | 0.298 | 0.300 | 0.301 | 0.301 | 0.303 | 0.305 | 0.310 |

where RT = retention time; spec = Specification (as % Total Peak Area i.e. TPA %)

### Example 2-Purification

Scheme 6 below shows the postulated mechanisms involving formation of impurities through further reactions of methoxyflurane.

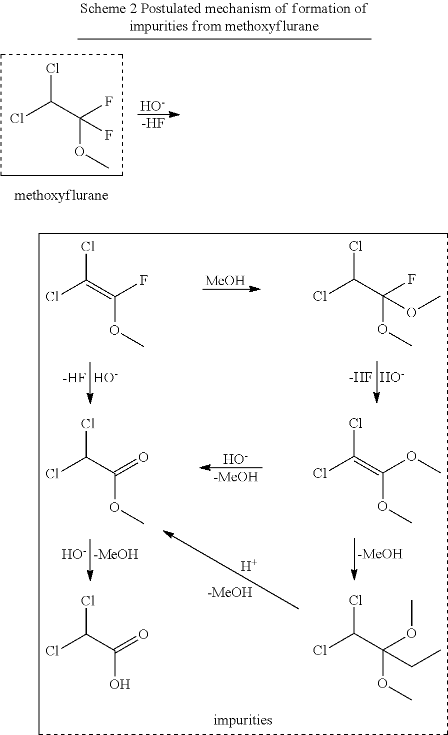

Impurities from the organic phase obtained by the procedure of Example 1 were mainly made by methoxyethene impurity. The impurity was removed from the crude methoxyflurane product in the organic phase as follows.

A 10% v/v methanesulfonic acid (MSA) solution (based on 2 L of MSA for 20 L crude methoxyflurane product) was slowly added to the crude methoxyflurane product (15.44 L obtained in Example 1) in a controlled manner (as the reaction is potentially exothermic). An initial reaction temperature of 35° C.±5° C. was obtained. The mixture was stirred for approximately 45 minutes after which time the methoxyethene (vinyl ether) impurity was no longer detected by gas chromatography.

### Example 3

Using an apparatus setup of the kind described in Example 1, a number of reactions were performed using different parameters, as described below. Specific parameters of the flow reactor and respective tubular flow lines were as follows.

Total internal volume of tubular flow lines: 100 ml

Individual tubular flow line diameter (internal): ¼ inch (6.35 mm)

Tubular flow lines arrangement: 4×3 linear array

Static mixer (located in the flow reactor): Yes

Base/MeOH solution pump cooling jacket: Yes <15° C.

DCDFE pump cooling jacket: Yes <15° C.

Process parameters and composition of the reactor effluent, determined by gas chromatography performed on the reactor effluent, are detailed in Table 3 below.

TABLE 3
Percentage (%) methoxyflurane produced under different reaction conditions from
DCDFE (dichloro-difluoroethylene)

| | | | | | Total | | |
|---|---|---|---|---|---|---|---|
| | | DCDFE | MeOH | | flow | | Percentage (%) |
| | Reaction | Flow | Flow | | rate | | methoxyflurane |
| Exp. | Temp | rate | rate | Base | (mL/ | RT | as measured |
| No. | (° C.) | (mL/min) | (mL/min) | (in MeOH) | min) | (min) | by GC |
| 1 | 30 | 15 | 25 | KOH (2%) | 40 | 2.5 | 91.82 |
| 2 | 30 | 15 | 30 | KOH (2%) | 45 | 2.22 | 93.88 |
| 3 | 30 | 10 | 20 | KOH (2%) | 30 | 3.33 | 93.08 |
| 4 | 1 | 10 | 20 | KOH (2%) | 30 | 3.33 | 93.61 |
| 5 | 1 | 6.25 | 12.5 | KOH (2%) | 18.75 | 5.33 | 95.67 |
| 6 | 0 | 5 | 20 | KOH (5%) | 25 | 4 | 94.88 |
| 7 | −5 | 5 | 20 | KOH (5%) | 25 | 4 | 95.86 |
| 8 | 5 | 5 | 25 | NaOH | 30 | 3.33 | 94.02 |
| | | | | (2.125%) | | | |
| 9 | 0 | 5 | 25 | NaOH | 30 | 3.33 | 95.71 |
| | | | | (2.125%) | | | |
| 10 | 0 | 3.3 | 16.7 | NaOH | 20 | 5 | 96.63 |
| | | | | (2.125%) | | | |
| 11 | 0 | 4 | 20 | NaOH | 24 | 4.17 | 95.75 |
| | | | | (2.125%) | | | |
| 12 | 0 | 6 | 30 | NaOH | 36 | 2.78 | 93.49 |
| | | | | (2.125%) | | | |

where: Exp. No is experiment number; GC is gas chromatography; and RT is residence time in the conduit system.

### Example 4

In addition to treatment of the crude methoxyflurane product with a 10% v/v methanesulfonic acid (MSA) solution to remove the methoxyethene (vinyl ether) impurity (as per Example 2, Step (A)), the following agents were shown to be similarly effective.

Hydrogen Chloride (37%)

An approximate 50% v/v solution (i.e. to crude methoxyflurane product) of HCl (37%) was added to the crude methoxyflurane product and monitored over the course of approximately 3 hours 45 minutes after which time the methoxyethene (vinyl ether) impurity was essentially no longer detectable by gas chromatography i.e. 0.01% vinyl impurity.

Time 0 min: 5.96% vinyl ether impurity

Time 1 h 18 min: 0.15% vinyl ether impurity

Time 2 h: 0.05% vinyl ether impurity

Time 3 h 45 min: 0.01% vinyl ether impurity

Bleach

An approximate 5.6% v/v solution of bleach (relative to the volume of crude methoxyflurane product) was added to the crude methoxyflurane product and stirred for approximately 72 hours after which time the methoxyethene (vinyl ether) impurity was essentially no longer detectable by 20 gas chromatography i.e. 0.01% vinyl impurity.

Oxone With and Without Water

An approximate 33% v/v aqueous solution of 4 g oxone/10 mL water (i.e. to 20 mL crude methoxyflurane product) was added to the crude methoxyflurane and stirred over the course of approximately 2.5 hours after which time the methoxyethene (vinyl ether) impurity was essentially no longer detectable by gas chromatography.

Time 0: 0.9% vinyl ether impurity

Time 1 h: 0.002% vinyl ether impurity

Time 2.5 h: 0.001% vinyl ether impurity

In a separate test, an approximate 33% v/v aqueous solution of 2 g oxone/10 mL water (i.e. to 20 mL crude methoxyflurane product) was added to the crude methoxyflurane and stirred over the course of approximately 2.5 hours after which time the methoxyethene (vinyl ether) impurity was essentially no longer detectable by gas chromatography.

Time 0: 0.9% vinyl ether impurity

Time 1 h: 0.1% vinyl ether impurity

Time 2.5 h: 5 0.06% vinyl ether impurity

In a separate test, an approximate 50% v/v aqueous solution of 4 g oxone/20 mL water (i.e. to 20 mL crude methoxyflurane product) was added to the crude methoxyflurane and stirred over the course of approximately 2 hours after which time the methoxyethene (vinyl ether) impurity was essentially no longer detectable by gas chromatography, i.e. 0.06%.

In a separate test, oxone (2 g) was added to the crude methoxyflurane (10 mL) and stirred over the course of approximately 3.25 hours after which time the methoxyethene (vinyl ether) impurity was essentially no longer detectable by gas chromatography, i.e. 0.03%.

### Example 5

A number of further synthesis tests were performed on the basis of the following parameters.

KOH/methanol solution: 2.5% KOH in Methanol; Flowrate 250 ml/min; Pre-cooled 0° C. to 5° C.

DCDFE: flowrate 50 ml/min; Pre-cooled 0° C. to 5° C.

Water: Water (potable); Flowrate 250 ml/min; room temperature

Total internal volume of tubular flow lines: approximately 200 mL-350 ml (for example 350 ml)

Individual tubular flow line diameter (internal): approximately 2 to 10 mm (for example 4.5 mm)

Individual tubular flow line length: approximately 12 m to 21 m (for example 21 m)

Tubular flow line arrangement: Coil

Total flow rate within flow reactor (e.g. effluent flow rate): approximately 50 ml/min to 500 ml/min (for example 300 ml/min)

Residence time inflow reactor: approximately >0 mins but <5 mins (for example >1 min but <2 mins, for example approximately 1 minute) Static mixer (located within the flow reactor): No

Shell (external casing of flow reactor) jacket cooling: 0° C. to 45° C. (for example cooled to 5° C. or below)

Cooling liquid: 15% glycol in water

Details of the procedure adopted for the purpose of this example are described below.

The KOH/methanol solution and DCDFE were pumped to mixing point (T-section), where they mixed before entering the tubular flow lines. The line pressure within the tubular flow lines was recorded at a range of 1.5-3.5 bar during the course of reaction, and regulated by a back pressure regulator downstream of the flow reactor. The reactor effluent exiting the flow reactor was mixed with water and, and the mixture passed through a linear static mixer before being collected in a collection vessel/reservoir.

Three samples of the reactor effluent/water mixture (i.e. Samples 1, 2 and 3) downstream of the static mixer were analysed and the results are presented in Tables 5, 6 and 7 below (corresponding to the gas chromatography spectrums shown in FIGS. 4, 5 and 6 respectively).

Sample 1 was collected for analysis as an “in-flow” sample of the crude reaction product after passage through the static mixer to provide a real-time analysis of the reaction progress in the conduit system at 15 minutes run time. Samples 2 and 3 were collected for analysis as a “bulk” sample of the crude reaction product after passage through the static mixer and collection in the collection vessel/reservoir to provide an accumulated average of the crude reaction product at 15 minutes and 30 minutes run time respectively. The results presented show that the purity/conversion ratio of the crude reaction product (methoxyflurane/DCDFE) was 97.431%/2.016% (Sample 1: in-flow, 15 mins), 96.956%/2.296% (Sample 2: collected bulk, 15 mins), and 97.989%/1.311% (Sample 3, collected bulk, 30 mins).

TABLE 5
Gas chromatography analysis of in-flow sample of crude reaction product collected at 15
minutes run time (Sample 1)

| | RT | RRT | Height | Area | |
|---|---|---|---|---|---|
| Name | (min) | Index | (μV) | (μV.Min) | Area % |
| Methanol | 2.30 | 0.52 | 456.3 | 14.8 | 0.022 |
| DCDFE | 2.46 | 0.55 | 33803.2 | 1347.9 | 2.016 |
| Halomar | 3.04 | 0.68 | 307.1 | 11.0 | 0.016 |
| Unknown | 3.20 | 0.72 | 183.6 | 9.4 | 0.014 |
| Methoxyflurane | 4.45 | 1.00 | 737411.0 | 65126.3 | 97.431 |
| Methoxyethene | 4.65 | 1.05 | 9542.1 | 334..0 | 0.500 |
| TOTAL | | | | | 100.000 |

where RT = Retention Time; and RRT = Relative Retention Time

TABLE 6
Gas chromatography analysis of “collected bulk” sample of crude reaction product collected
at 15 minutes run time (Sample 2)

| | RT | RRT | Height | Area | |
|---|---|---|---|---|---|
| Name | (min) | Index | (μV) | (μV.Min) | Area % |
| Methanol | 2.29 | 0.52 | 3962.7 | 117.2 | 0.208 |
| DCDFE | 2.45 | 0.56 | 22969.9 | 1294.4 | 2.296 |
| Halomar | 3.03 | 0.69 | 245.8 | 9.6 | 0.017 |
| Chloroform | 3.18 | 0.72 | 144.7 | 7.7 | 0.014 |
| Methoxyflurane | 4.41 | 1.00 | 680294.3 | 54668.3 | 96.956 |
| Methoxyethene | 4.63 | 1.05 | 8068.8 | 287.4 | 0.510 |
| TOTAL | | | | | 100.000 |

where RT = Retention Time; and RRT = Relative Retention Time

TABLE 7
Gas chromatography analysis of “collected bulk” sample of crude reaction product at 30
minutes run time (Sample 3)

| | RT | RRT | Height | Area | |
|---|---|---|---|---|---|
| Name | (min) | Index | (μV) | (μV.Min) | Area % |
| Methanol | 2.30 | 0.52 | 22262.9 | 76.8 | 0.145 |
| DCDFE | 2.49 | 0.56 | 692.4 | 1.311 | 0.79 |
| Halomar | 3.04 | 0.69 | 214.8 | 9.4 | 0.018 |
| Unknown | 3.19 | 0.72 | 126.9 | 6.7 | 0.013 |
| Chloroform | 3.27 | 0.74 | 32.4 | 1.4 | 0.003 |
| Methoxyflurane | 4.41 | 1.00 | 653342.8 | 51767.3 | 97.989 |
| Methoxyethene | 4.63 | 1.05 | 7378.7 | 275.6 | 0.522 |
| TOTAL | | | | | 100.000 |

where RT = Retention Time; and RRT = Relative Retention Time

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Many modifications will be apparent to those skilled in the art without departing from the scope of the present invention.

1. A process for continuous preparation of halogenated alkoxyethane of general formula XClHC—CF₂OR, where X is —Cl or —F and OR is C₁₋₄ alkoxy, the process comprising a step of introducing in a flow reactor reaction components comprising (i) a compound of general formula XClC═CF₂, (ii) a base, and (iii) a C₁₋₄ alkanol, wherein
a) the flow reactor comprises one or more tubular flow line(s) having an internal cross-sectional area of less than 115 mm² through which the reaction components flow as a reaction mixture, and
b) the halogenated alkoxyethane is formed at least upon the reaction components mixing, with the so formed halogenated alkoxyethane flowing out of the flow reactor in a reactor effluent.

2. The process of claim 1, wherein the one or more tubular flow line(s) has/have an internal cross-sectional area of less than 30 mm².

3. The process of claim 1 or 2, wherein the one or more tubular flow line(s) has/have a circular internal cross-section.

4. The process of claim 3, wherein the one or more tubular flow line(s) has/have a diameter between 0.1 and 6 mm.

5. The process of any one of claims 1-4, wherein the one or more tubular flow line(s) are provided in a coiled arrangement.

6. The process of any one of claims 1-5, wherein the one or more tubular flow line(s) has/have a total internal volume of at least 100 ml.

7. The process of any one of claims 1-6, wherein the reactor effluent containing the halogenated alkoxyethane exits the reactor at a flow rate of at least 50 ml/min.

8. The process of any one of claims 1-7, wherein the reactor effluent contains at least 90% by volume of the halogenated alkoxyethane.

9. The process of any one of claims 1-8, wherein the flow reactor provides a residence time of less than about 5 minutes.

10. The process of any one of claims 1-9, wherein the reaction mixture is obtained by mixing (i) a solution of the C₁₋₄ alkanol and the base with (ii) the compound of general formula XClC═CF₂.

11. The process of any one of claims 1-10, wherein the mixing is performed by combining (i) a flow of the solution of the C₁₋₄ alkanol and the base with (ii) a flow of the compound of general formula XClC═CF₂ according to a flow-rate ratio from 1:1 to 10:1.

12. The process of any one of claims 1-11, wherein the base is used in an amount of between 1% and 30% by weight relative to the total weight of base and C₁₋₄ alkanol.

13. The process of any one of claims 1-12, wherein the compound of general formula XClC═CF₂ is Cl₂C═CF₂ or FClC═CF₂.

14. The process of any one of claims 1-13, wherein the C₁₋₄ alkanol is selected from methanol (CH₃OH), ethanol (CH₃CH₂OH), 1-propanol (CH₃CH₂CH₂OH), 2-propanol ((CH₃)₂CHOH), 1-butanol (CH₃CH₂CH₂CH₂OH), 2-butanol (CH₃CH₂CHOHCH₃), 2-methyl-1-propanol ((CH₃)₂CHCH₂OH), 2-methyl-2-propanol ((CH₃)₃COH), and a combination thereof.

15. The process of any one of claims 1-14, wherein the halogenated alkoxyethane is Cl₂HC—CF₂OCH₃ (methoxyflurane) or ClFHC—CF₂OCH₃.

16. The process of any one of claims 1-15, wherein the base comprises an alkali metal base cation or an ammonium base cation.

17. The process of any one of claims 1-16, wherein the base is used in substoichiometric amount relative to the compound of general formula XClC═CF₂.

18. The process of any one of claims 1-17, wherein the base is selected from sodium hydroxide, potassium hydroxide, sodium methoxide, potassium methoxide, tetrabutylammonium hydroxide, benzyl(trimethyl)ammonium hydroxide, N-methyl-N,N,N-trioctylammonium chloride, tetraethylammonium hydroxide, tetramethylammonium hydroxide, and tetramethylphosphonium hydroxide.

19. The process of any one of claims 1-18, wherein water is added to the reactor effluent.

20. The process of any one of claims 1-19, further comprising a purification step in which the reactor effluent, or an organic phase derived from the reactor effluent and containing the halogenated alkoxyethane, is treated with an acid.

21. The process of any one of claims 1-19, further comprising a purification procedure that comprises, in the following order, the steps of:
a) mixing the reactor effluent with a polar solvent to induce phase separation between a polar phase and an organic phase,
b) separating the organic phase from the polar phase,
c) treating the separated organic phase with an amine,
d) washing the organic phase obtained in c) with an acid solution,
e) desiccating the organic phase obtained in d), and
f) distilling the organic phase obtained in e) to retrieve a purified distillate comprising the halogenated alkoxyethane.

22. The process of claim 20 or 21, wherein the acid is selected from hydrochloric acid, sulfuric acid, sulphurous acid, methanesulfonic acid, trifluoromethanesulfonic acid, phosphoric acid, acetic acid, trifluoroacetic acid, nitric acid, nitrous acid, hypochlorous acid, chlorous acid, chloric acid, perchloric acid, and a combination thereof.

23. The process of claim 20 or 21, wherein the acid is methanesulfonic acid.
